(19) United States
(12) Patent Application Publication
(10) Pub. No.: US 2021/0324010 A1
(43) Pub. Date: Oct. 21, 2021

(54) FUSION PRODUCTS AND BIOCONJUGATES CONTAINING MIXED CHARGE PEPTIDES

(71) Applicant: University of Washington, Seattle, WA (US)

(72) Inventors: Caroline Tsao, Seattle, WA (US); Sijin Luozhong, Seattle, WA (US); Trevor Corrigan, Seattle, WA (US); Shaoyi Jiang, Seattle, WA (US); Erik Liu, Seattle, WA (US); Patrick McMullen, Seattle, WA (US)

(73) Assignee: University of Washington, Seattle, WA (US)

(21) Appl. No.: 17/283,708

(22) PCT Filed: Oct. 10, 2019
(86) PCT No.: PCT/US2019/055703

§ 371 Date: Apr. 8, 2021

Related U.S. Application Data
(60) Provisional application No. 62/743,663, filed on Oct. 10, 2018.

(51) Int. Cl. C07K 14/00; A61K 47/64; C07K 14/535; C07K 14/56; C07K 14/435

(52) U.S. Cl. CPC C07K 14/00; A61K 47/645; C07K 2319/00; C07K 14/56; C07K 14/43595; C07K 14/535

(57) ABSTRACT

Charged polypeptides, their conjugates, and fusion proteins comprising such polypeptides are disclosed. Inclusion of such a polypeptide in a fusion protein increases the protein's properties such as stability and circulation half-life, which results in a better therapeutic efficacy compared to an active protein alone. Thus, a fusion protein or a conjugate of the disclosure can be useful in developing a protein or peptide drug, treating or preventing diseases, disorders, or conditions, or improving a subjects health or wellbeing.

### CROSS-REFERENCE(S) TO RELATED APPLICATION(S)

This application claims the benefit of U.S. Patent Application No. 62/743,663, filed Oct. 10, 2018, which is expressly incorporated herein by reference in its entirety.

### STATEMENT OF GOVERNMENT LICENSE RIGHTS

This invention was made with Government support under Grant No. HDTRA1-13-1-0044 P00011 awarded by the Defense Threat Reduction Agency (DTRA). The Government has certain rights to this invention.

### STATEMENT REGARDING SEQUENCE LISTING

The sequence listing associated with this application is provided in text format in lieu of a paper copy and is hereby incorporated by reference into the specification. The name of the text file containing the sequence listing is 70421_Sequence_final_2019-10-10. The text file is 60.1 KB; was created on Oct. 10, 2019; and is being submitted via EFS-Web with the filing of the specification.

### BACKGROUND

Peptides and proteins are known to have great therapeutic potential against many diseases and syndromes. Progress in the field of pharmaceutical biotechnology has increased the value and number of protein- and peptide-based therapeutics in the market. Currently, more than 100 proteins have been approved as therapeutics, with many more undergoing clinical trials. Despite current and future growth of the biopharmaceutical market, there are significant challenges relating to implementing promising therapeutic proteins. Many of these challenges greatly decrease the efficacy of therapeutics, and these limitations are often imparted through properties inherent to therapeutics and their manufacturing. These inherent properties can lead to conformational changes, degradation, aggregation, precipitation, and adsorption onto surfaces. Additionally, these therapeutic proteins are often characterized by short half-lives and immunogenic responses, particularly considering that many of these recombinant proteins are either sourced from non-human organisms or are expressed in a non-human host. The resulting poor pharmacokinetics has been a key issue facing biopharmaceutical development.

Currently one of the most accepted methods is the use of polyethylene glycol (PEG), a non-toxic and putatively non-immunogenic polymer, in modifying therapeutic proteins. The process, commonly known as PEGylation, is known to change the physical and chemical properties of the biomolecule, including conformation, electrostatic binding, and hydrophobicity, and can result in improved pharmacokinetic properties for the drug. Advantages of PEGylation include improvements in drug solubility and reduction of immunogenicity, increased drug stability and circulation time once administered, and reductions in proteolysis and renal excretion, all of which allow for reduced dosing frequency leading to increased patient compliance and better therapeutic outcomes. PEGylation technology has been applied to a number of therapeutic proteins to provide new drugs that have been approved by the U.S. FDA. However, concerns remain about the usage of PEGylated biopharmaceuticals due to induced and pre-existing anti-PEG antibodies. PEGylated proteins have demonstrated the ability to elicit immune responses from some healthy individuals with the presence of anti-PEG antibodies. Injection of an antigenic substance can potentially cause a cytokine cascade or other potentially severe immune responses and as such should be avoided as a component of medical formulations. Previously, it was demonstrated that the use of zwitterionic polymers such as poly(carboxybetaine) (pCB) as an alternative to amphiphilic PEG imparts superhydrophilic, ultra-low biofouling, and protein-stabilizing characteristics. The chemical conjugation of pCB to proteins increases their stability without affecting their activity. However, pCBs, due to their synthetic origin, can suffer from the same drawbacks as PEG. Finally, it has been demonstrated that incorporation of a domain consisting of repeating lysine (K) and glutamic acid (E) residues into a fusion polypeptide can improve certain properties of the resulting polypeptide; however, it is hard to control the size and shape of such polypeptides.

A need exists for pharmaceutical agents, such as proteins, with better pharmacokinetics and other advantageous properties, including improved solubility, reduced dosage frequency, extended circulating life, increased stability, and enhanced protection from proteolytic degradation.

### DESCRIPTION OF THE DRAWINGS

The foregoing aspects and many of the attendant advantages of this invention will become more readily appreciated as the same become better understood by reference to the following detailed description, when taken in conjunction with the accompanying drawings.

FIG. 1 is a photograph of a Western Blot of MBP-EKX-GCSF variants after purification using IMAC column. Protein transferred to polyvinylidene difluoride (PVDF) membrane and probed using monoclonal anti-GCSF antibody (Invitrogen). Bands from lane 2 to 9 indicates the presents of MBP-EKX-GCSF.

FIG. 2A is a Circular Dichroism (CD) profile of EKX-GCSF variants obtained in 10 mM Potassium Phosphate pH 8 with 1 μM of EKX-GCSF or GCSF where indicated.

FIG. 2B shows GCSF CD profile subtracted from EKX-GCSF variants to obtain EKX component of CD profile.

FIG. 3 is a graph of serum concentration profiles of EKX-GCSF and GCSF alone. EKX-GCSF or GCSF (20 nmol/kg) were injected into C57BL/6 Mice (6 weeks old) by retroorbital injection. Blood was drawn and analyzed for EKX-GCSF or GCSF using ELISA assay.

FIG. 4A is a graph of normalized serum concentration profiles of EKX-GCSF and GCSF alone. EKX-GCSF or GCSF (10 nmol/kg) were injected into Sprague-Dawley rats via tail vein injection. Blood was drawn and analyzed for EKX-GCSF or GCSF using ELISA assay.

FIG. 4B is a graph of white blood cell count from animals injected with 10 nmol/kg EKP-GCSF, EK-GCSF, and GCSF at indicated time points. White blood cell count determined by Medix LeukoTic Bluplus WBC test kit.

FIG. 5 is a photograph of an SDS-PAGE gel of EKP-hIFNα2a, EK-hIFNα2a and hIFNα2a alone expressed and secreted from HEK293F cell. Purification was performed using HA purification kit (ThermoFisher).

FIG. 6 is a graph of serum concentration profiles of EKP-hIFNα2a (EKP-hIFNα2a), EK-hIFNα2a (EK-hIFNα2a), and hIFNα2a alone (hIFNα2a). EKP-hIFNα2a, EK-hIFNα2a, and hIFNα2a alone (50 nmol/kg) were injected via retro-orbital method into C57BL/6 mice (6 weeks old). Blood was drawn at indicated time points and analyzed for EKP-hIFNα2a and hIFNα2a using ELISA assay. The dashed line is indicating that the concentration was below detection limit (˜40 ng/mL).

FIG. 7 is a photograph of an SDS-PAGE gel of purified eGFP and EKX-eGFP variants with ladder and lanes as indicated.

### SUMMARY

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

In one aspect, provided herein is a polypeptide comprising:

a) a plurality of negatively charged amino acids;

b) a plurality of positively charged amino acids; and

c) a plurality of additional amino acids independently selected from the group consisting of proline, serine, threonine, asparagine, glutamine, glycine, and derivatives thereof; and wherein the ratio of the number of positively charged amino acids to the number of positively charged amino acids is from about 1:0.5 to about 1:2.

In some embodiments, the plurality of negatively charged amino acids is independently selected from the group consisting of aspartic acid, glutamic acid, and derivatives thereof. In some embodiments, the plurality of positively charged amino acids is independently selected from the group consisting of lysine, histidine, arginine, and derivatives thereof.

In some embodiments, the positively charged amino acids and negatively charged amino acids constitute from about 20% to about 95%, from about 30% to about 95%, about 40% to about 95%, about 50% to about 95%, about 40% to about 90%, about 50% to about 90%, from about 40% to about 80%, or from about 50% to about 70% of the total number of amino acids present in the charged domain.

In some embodiments, the polypeptide comprises from about 6 to about 1000 amino acids, from about 20 to about 1000 amino acids, from about 30 to about 1000 amino acids, from about 50 to about 1000 amino acids, from about 80 to about 1000 amino acids or from about 80 to about 600 amino acids.

In some embodiments, the ratio of positively charged amino acids to negatively charged amino acids is from about 1:07 to about 1:1.4, from about 1:0.8 to about 1:1.25, or from about 1:0.9 to about 1:1.1.

In some embodiments, the polypeptide comprises at least two pairs comprising a positively charged amino acid adjacent to a negatively charged amino acid. In some embodiments, the polypeptide comprises a random sequence. In some embodiments, the polypeptide is substantially electronically neutral.

In some embodiments, the polypeptide comprises a plurality of lysines and a plurality of negatively charged amino acids selected from the group consisting of glutamic acid and aspartic acid. In some embodiments, the polypeptide comprises a plurality of histidines and a plurality of negatively charged amino acids selected from the group consisting of glutamic acid and aspartic acid.

In some embodiments, the plurality of additional amino acids is selected from the group consisting of serine, asparagine, glycine, and proline. In some embodiments, the plurality of additional amino acids is selected from the group consisting of serine, glycine, and proline.

In some embodiments, the plurality of additional amino acids is selected from the group consisting of serine and glycine. In some embodiments, the plurality of additional amino acids is prolines. In some embodiments, the plurality of additional amino acids is glycines. In some embodiments, the plurality of additional amino acids is serines.

In some embodiments, the polypeptide comprises a plurality of lysines, a plurality of glutamic acids, and a plurality of additional amino acids selected from the group consisting of serine, glycine, and proline.

In some embodiments, the polypeptide comprises a plurality of lysines, a plurality of glutamic acids, and a plurality of additional amino acids selected from the group consisting of glycine and proline.

In some embodiments, the polypeptide is substantially electronically neutral at pH of about 7.4.

In another aspect, provided herein is a bioconjugate comprising at least one polypeptide disclosed herein covalently coupled to a biomolecule.

In another aspect, provided herein is a method of stabilizing a biomolecule, comprising conjugating one or more polypeptides disclosed herein to a biomolecule.

In some embodiments, the biomolecule is a polypeptide, a synthetic polymer, a nucleic acid, a glycoprotein, a proteoglycan, a fluorescent dye, a small molecule, a fatty acid, or a lipid.

In another aspect, provided herein is a fusion protein comprising one or more functional domains linked to one or more charged domains, wherein the one or more charged domains comprises a polypeptide disclosed herein.

In another aspect, provided herein is a nucleic acid comprising a sequence encoding the fusion protein disclosed herein.

In another aspect, provided herein is an expression vector comprising the nucleic acid disclosed herein.

In another aspect, provided herein is a cell comprising the nucleic acid or expression vector disclosed herein. In some embodiments, the cell is a prokaryotic cell or eukaryotic cell.

In another aspect, provided herein is a method of preparing a fusion protein, comprising expressing the expression vector disclosed herein.

In some embodiments, the method further comprises isolating the polypeptide. In some embodiments, the isolating the polypeptide comprises a method selected from the group consisting of protein precipitation, size exclusion chromatography, affinity chromatography, separation based on electrostatic properties, separation based on hydrophilic or hydrophobic properties, separation based on matrix-free electrophoresis techniques, or a combination thereof.

### DETAILED DESCRIPTION

Disclosed herein are polypeptides, their bioconjugates, and fusion proteins comprising such polypeptides, wherein the polypeptides comprise a plurality of amino acids independently selected from negatively charged amino acids, a plurality of amino acids independently selected from positively charged amino acids, and a plurality of amino acids independently selected from neutral hydrophilic amino acids and proline. Conjugates of biomolecules with the polypeptides and fusion proteins comprising the polypeptides can have reduced immunogenicity, increased half-life, increased yield, and/or improved specific targeting compared to the parent non-modified molecule.

Polypeptides

In one aspect, provided herein is a polypeptide comprising:

a) a plurality of negatively charged amino acids;

b) a plurality of positively charged amino acids; and

c) a plurality of additional amino acids independently selected from the group consisting of proline, serine, threonine, asparagine, glutamine, glycine, and derivatives thereof; and

wherein the ratio of the number of positively charged amino acids to the number of positively charged amino acids is from about 1:0.5 to about 1:2.

As used herein, the term “amino acid” encompasses both individual amino acids and amino acid residues incorporated into a polypeptide chain. It is understood that when the term “amino acid” is mentioned in the context of a polypeptide, the term refers to an amino acid linked to one or two adjacent amino acids by peptide bonds. As used herein, the term “about” means+5% of the stated value.

Negatively charged amino acids include amino acids comprising a group that can be negatively charged, such as a carboxylic acid group, as well as their derivatives and latent negatively charged groups. As used herein, “latent negatively charged group” is a functional group, such as an ester, that can be converted to negatively charged group, such as a carboxylic acid, when exposed to an appropriate environmental stimulus. Positively charged amino acids include amino acids comprising a group that can be positively charged, such as amino group, as well as their derivatives and latent positively charged groups. As used herein, “latent positively charged group” is a functional group, such as a t-butyloxycarbonyl-(t-Boc) protected amino group, that can be converted to a positively charged group, such as amino group, when exposed to an appropriate environmental stimulus.

In some embodiments of the polypeptides disclosed herein, the plurality of negatively charged amino acids is independently selected from the group consisting of aspartic acid, glutamic acid, and derivatives thereof. In certain embodiments, the plurality of positively charged amino acids is independently selected from the group consisting of lysine, histidine, arginine, and derivatives thereof.

In some embodiments, positively charged amino acids and negatively charged amino acids constitute from about 20% to about 95%, from about 30% to about 95%, from about 40% to about 95%, from about 50% to about 95%, from about 40% to about 90%, from about 50% to about 90%, from about 40% to about 80%, or from about 50% to about 70% of the total number of amino acids present in the polypeptide. In some embodiments, the positively charged amino acids constitute from about 10% to about 48%, from about 15% to about 48%, from 20% to about 48%, from about 25% to about 48%, from about 20% to about 45%, from about 25% to about 45%, from about 20% to about 40%, or from about 25% to about 35% of the total number of amino acids present in the polypeptide. In some embodiments, the negatively charged amino acids constitute from about 10% to about 48%, from about 15% to about 48%, from 20% to about 48%, from about 25% to about 48%, from about 20% to about 45%, from about 25% to about 45%, from about 20% to about 40%, or from about 25% to about 35% of the total number of amino acids present in the polypeptide.

The polypeptides disclosed herein typically comprise from about 6 to about 1000 amino acids, from about 20 to about 1000 amino acids, from about 30 to about 1000 amino acids, from about 50 to about 1000 amino acids, from about 80 to about 1000 amino acids, from about 80 to about 600 amino acids, or from about 50 to about 500 amino acids.

The polypeptides disclosed herein comprise negatively charged amino acids and positively charged amino acids in substantially equal numbers. In some embodiments, the ratio of the number of negatively charged amino acids to the number of positively charged amino acids is from about 1:0.5 to about 1:2, from about 1:07 to about 1:1.4, from about 1:0.8 to about 1:1.25, or from about 1:0.9 to about 1:1.1. Thus, the polypeptides disclosed herein are substantially electronically neutral. As used herein, the term “substantially electronically neutral” refers to the property of a polypeptide having a net charge of substantially zero (i.e., a polypeptide with about the same number of positively charged amino acids and negatively charged amino acids). In some embodiments, the polypeptide is substantially electronically neutral at pH f about 7.4.

In some embodiments, the polypeptide comprises a plurality of lysines and a plurality of negatively charged amino acids selected from the group consisting of glutamic acid and aspartic acid. In some embodiments, the polypeptide comprises a plurality of histidines and a plurality of negatively charged amino acids selected from the group consisting of glutamic acid and aspartic acid.

In some embodiments of the polypeptides disclosed herein, the plurality of additional amino acids is selected from the group consisting of serine, asparagine, glycine, and proline. Polypeptides of the disclosure can comprise only one type of additional amino acid (e.g., proline), two different additional amino acids (e.g., proline and glycine), three different additional amino acids (e.g, serine, glycine, and proline). In some embodiments, the polypeptides comprise one additional amino acid. In some embodiments, the polypeptides comprise two additional amino acids.

In some embodiments of the polypeptides disclosed herein, the plurality of additional amino acids is selected from the group consisting of serine, glycine, and proline. In some embodiments of the polypeptides disclosed herein, the plurality of additional amino acids is selected from the group consisting of serine and glycine.

In some embodiments of the polypeptides disclosed herein, the plurality of additional amino acids is two or more prolines. In some embodiments of the polypeptides disclosed herein, the plurality of additional amino acids is two or more glycines. In some embodiments of the polypeptides disclosed herein, the plurality of additional amino acids is two or more serines.

In some embodiments, the polypeptide comprises a plurality of lysines, a plurality of glutamic acids, and a plurality of additional amino acids selected from the group consisting of serine, glycine, and proline.

In some embodiments, the polypeptide comprises a plurality of lysines, a plurality of glutamic acids, and a plurality of additional amino acids selected from the group consisting of glycine and proline.

In some embodiments, the polypeptide consists essentially of a plurality of negatively charged amino acids; a plurality of positively charged amino acids; and a plurality of additional amino acids independently selected from the group consisting of proline, serine, threonine, asparagine, glutamine, glycine, and derivatives thereof, and optionally an affinity tag, such a histidine tag which can be used for affinity purification of the polypeptide. In some embodiments, the polypeptide consists essentially of a plurality of glutamic acids; a plurality of lysines; and a plurality of additional amino acids independently selected from the group consisting of proline and glycine, and optionally an affinity tag, such a histidine tag which can be used for affinity purification of the polypeptide.

The amino acids in the polypeptides of the disclosure can be arranged in any manner or sequence. In some embodiments, the polypeptide comprises at least two pairs of a positively charged amino acid adjacent to a negatively charged amino acid. In some embodiments, the polypeptide comprises at least three pairs of a positively charged amino acid adjacent to a negatively charged amino acid. In some embodiments, the polypeptide comprises at least five pairs of a positively charged amino acid adjacent to a negatively charged amino acid. In some embodiments, the polypeptide comprises at least ten pairs of a positively charged amino acid adjacent to a negatively charged amino acid. In some embodiments, the polypeptide comprises a random sequence. For example, when a polypeptide of the disclosure comprises a plurality of glutamic acids (E), a plurality of lysines (K), and a plurality of glycines (G), the polypeptide can comprise a sequence comprising an EKG tri-peptide as a repeating unit, e.g., (EKG)ₙ, wherein n is two or greater. In some embodiments, the exemplary polypeptide comprising a plurality of glutamic acids (E), a plurality of lysines (K), and a plurality of glycines (G) can have a random sequence, such as EKGGKEGKKEEEGG . . . . In some embodiments, the polypeptides do not comprise blocks of five or more identical amino acids.

In some embodiments, the polypeptide is a random coil polypeptide, i.e., the polypeptide adopts/forms random coil conformation, for example, in aqueous solution or at physiological conditions. The term “physiological conditions” refers to those conditions in which proteins usually adopt their native, folded conformation. In some embodiments, the random coil conformation mediates an increased in vivo and/or in vitro stability of the polypeptide or a bioconjugate thereof, such as the in vivo and/or in vitro stability in biological samples or in physiological environments.

The polypeptides disclosed herein can be prepared according to the methods known in the art, such as chemical peptide synthesis or cloning.

### Bioconjugates

In a second aspect, provided herein is a bioconjugate comprising at least one polypeptide disclosed herein, wherein the polypeptide is covalently coupled to a biomolecule. Suitable biomolecules include biopolymers (e.g., proteins, peptides, oligonucleotides, polysaccharides), lipids, and small molecules.

In some embodiments, the biomolecule is a polypeptide (e.g., a protein, an enzyme, a short peptide, an antibody or a fragment thereof, a structural protein, etc.), a synthetic polymer, a nucleic acid, a glycoprotein, a proteoglycan, a fluorescent dye, a small molecule, a fatty acid, or a lipid.

In some embodiments, the biomolecule is a protein or peptide. The terms “protein,” “polypeptide,” and “peptide” can be used interchangeably. In certain embodiments, peptides range from about 5 to about 5000, 5 to about 1000, about 5 to about 750, about 5 to about 500, about 5 to about 250, about 5 to about 100, about 5 to about 75, about 5 to about 50, about 5 to about 40, about 5 to about 30, about 5 to about 25, about 5 to about 20, about 5 to about 15, or about 5 to about 10 amino acids in size, can contain L-amino acids, D-amino acids, or both, and can contain any of a variety of amino acid modifications or analogs known in the art. Such modifications include, e.g., terminal acetylation, amidation.

In some embodiments, the biomolecule can be a hormone, erythropoietin, insulin, cytokine, antigen for vaccination, or a growth factor. In some embodiments, the biomolecule can be an antibody and/or characteristic portion thereof. In some embodiments, antibodies can include, but are not limited to, polyclonal, monoclonal, chimeric (i.e., “humanized”), or single chain (recombinant) antibodies. In some embodiments, antibodies can have reduced effector functions and/or bispecific molecules. In some embodiments, antibodies may include Fab fragments and/or fragments produced by a Fab expression library (e.g. Fab, Fab′, F(ab′)₂, scFv, Fv, dsFv diabody, and Fd fragments.

In some embodiments, wherein a biomolecule is a protein, the polypeptide of the disclosure can be linked to the C or N terminus of the protein by a peptide bond.

In certain embodiments, the biomolecule is a nucleic acid (e.g., DNA, RNA, derivatives thereof). In some embodiments, the nucleic acid agent is a functional RNA. In general, a “functional RNA” is an RNA that does not code for a protein but instead belongs to a class of RNA molecules whose members characteristically possess one or more different functions or activities within a cell. It will be appreciated that the relative activities of functional RNA molecules having different sequences may differ and may depend at least in part on the particular cell type in which the RNA is present. Thus, the term “functional RNA” is used herein to refer to a class of RNA molecule and is not intended to imply that all members of the class will in fact display the activity characteristic of that class under any particular set of conditions. In some embodiments, functional RNAs include RNAi-inducing entities (e.g., short interfering RNAs (siRNAs), short hairpin RNAs (shRNAs), and microRNAs), ribozymes, tRNAs, rRNAs, RNAs useful for triple helix formation.

In some embodiments, the nucleic acid is a vector. As used herein, the term “vector” refers to a nucleic acid molecule (typically, but not necessarily, a DNA molecule) which can transport another nucleic acid to which it has been linked. A vector can achieve extra-chromosomal replication and/or expression of nucleic acids to which they are linked in a host cell. In some embodiments, a vector can achieve integration into the genome of the host cell. In some embodiments, vectors are used to direct protein and/or RNA expression. In some embodiments, the protein and/or RNA to be expressed is not normally expressed by the cell. In some embodiments, the protein and/or RNA to be expressed is normally expressed by the cell, but at lower levels than it is expressed when the vector has not been delivered to the cell. In some embodiments, a vector directs expression of any of the functional RNAs described herein, such as RNAi-inducing entities, ribozymes.

In some embodiments, the biomolecule is a carbohydrate. In certain embodiments, the carbohydrate is a carbohydrate that is associated with a protein (e.g. glycoprotein, proteogycan). Carbohydrates include both natural or synthetic carbohydrates. A carbohydrate can also be a derivatized natural carbohydrate. In certain embodiments, a carbohydrate can be a simple or complex sugar. In certain embodiments, a carbohydrate is a monosaccharide, including but not limited to glucose, fructose, galactose, and ribose. In certain embodiments, a carbohydrate is a disaccharide, including but not limited to lactose, sucrose, maltose, trehalose, and cellobiose. In certain embodiments, a carbohydrate is a polysaccharide, including but not limited to cellulose, microcrystalline cellulose, hydroxypropyl methylcellulose (HPMC), methylcellulose (MC), dextrose, dextran, glycogen, xanthan gum, gellan gum, starch, and pullulan. In certain embodiments, a carbohydrate is a sugar alcohol, including but not limited to mannitol, sorbitol, xylitol, erythritol, malitol, and lactitol.

In some embodiments, the biomolecule is a lipid. In certain embodiments, the lipid is a lipid that is associated with a protein (e.g., lipoprotein). Exemplary lipids include, but are not limited to, glycerides, monoglycerides, diglycerides, triglycerides, steroids (e.g., cholesterol, bile acids), vitamins (e.g., vitamin E), phospholipids, sphingolipids, and lipoproteins.

In some embodiments, the biomolecule is a fatty acid, e.g., an acid that has a long substituted or unsubstituted hydrocarbon chain (e.g., C5-050), including saturated and unsaturated chains. In some embodiments, the fatty acid can be one or more of caproic, caprylic, capric, lauric, myristic, palmitic, stearic, arachidic, behenic, or lignoceric acid. In some embodiments, the fatty acid can be one or more of palmitoleic, oleic, vaccenic, linoleic, alpha-linolenic, gamma-linoleic, arachidonic, gadoleic, arachidonic, eicosapentaenoic, docosahexaenoic, or erucic acid.

In some embodiments, the biomolecule is a small molecule and/or organic compound with pharmaceutical activity. In some embodiments, the biomolecule is a clinically-used drug. In some embodiments, the drug is an anti-cancer agent, antibiotic, anti-viral agent, anti-HIV agent, anti-parasite agent, anti-protozoal agent, anesthetic, anticoagulant, inhibitor of an enzyme, steroidal agent, steroidal or non-steroidal anti-inflammatory agent, antihistamine, immunosuppressant agent, anti-neoplastic agent, antigen, vaccine, antibody, decongestant, sedative, opioid, analgesic, anti-pyretic, birth control agent, hormone, prostaglandin, progestational agent, anti-glaucoma agent, ophthalmic agent, anti-cholinergic, analgesic, anti-depressant, anti-psychotic, neurotoxin, hypnotic, tranquilizer, anti-convulsant, muscle relaxant, anti-Parkinson agent, anti-spasmodic, muscle contractant, channel blocker, miotic agent, anti-secretory agent, anti-thrombotic agent, anticoagulant, anti-cholinergic, .beta.-adrenergic blocking agent, diuretic, cardiovascular active agent, vasoactive agent, vasodilating agent, anti-hypertensive agent, angiogenic agent, modulators of cell-extracellular matrix interactions (e.g., cell growth inhibitors and anti-adhesion molecules), inhibitor of DNA, RNA, or protein synthesis. In certain embodiments, a small molecule agent can be any drug. In some embodiments, the drug is one that has already been deemed safe and effective for use in humans or animals by the appropriate governmental agency or regulatory body, such as specific drugs disclosed in “Pharmaceutical Drugs: Syntheses, Patents, Applications” by Axel Kleemann and Jurgen Engel, Thieme Medical Publishing, 1999, and “The Merck Index: An Encyclopedia of Chemicals, Drugs, and Biologicals, Budavari et al. (eds.), CRC Press, 1996, both of which are incorporated herein by reference.

The polypeptide of the disclosure can be conjugated to the biomolecule by covalent coupling according to the methods known in the art. In some embodiments, the bioconjugate comprises two or more polypeptides of the disclosure covalently linked to a biomolecule. Both side chain groups and terminal groups of the polypeptides of the disclosure can be used to conjugate the polypeptide to the biomolecule. Likewise, the polypeptide can be attached to the biomolecule in any suitable manner, for example, to a side chain of a protein or a reactive group incorporated into a base of a nucleic acid.

In another aspect, provided herein is a method of stabilizing a biomolecule, comprising conjugating one or more polypeptides disclosed herein to a biomolecule. As used herein, “stabilizing a biomolecule” includes reducing the immunogenicity, increasing its biological half-life, and/or improved specific tissue or organ targeting as compared to the parent non-modified biomolecule.

### Fusion Proteins

In another aspect, provided herein is a fusion protein comprising one or more functional domains linked to one or more charged domains, wherein the one or more charged domains comprises:

a) a plurality of negatively charged amino acids;

b) a plurality of positively charged amino acids; and

c) a plurality of additional amino acids independently selected from the group consisting of proline, serine, threonine, asparagine, glutamine, glycine, and derivatives thereof; and

wherein the ratio of the number of positively charged amino acids to the number of positively charged amino acids is from about 1:0.5 to about 1:2.

As used herein, a “fusion protein” is a protein consisting of at least two domains that are encoded by separate genes that have been joined so that they are transcribed and translated as a single unit, producing a single polypeptide. In some embodiments, the domains of the fusion protein disclosed herein are contained with a single primary sequence of the protein, e.g., as a singular polypeptide.

As used herein, the term “functional domain” relates to any region or part of an amino acid sequence that is capable of autonomously adopting a specific structure and/or function. In some embodiments, the fusion protein as described herein can comprise at least one functional domain which can mediate a biological activity, which itself can be a fusion protein. The fusion proteins of the disclosure comprise at least one domain/part having and/or mediating biological activity and at least one charged domain. The fusion proteins of the invention also can consist of more than two domains and can comprise a spacer structure between the two domains or an additional domain, e.g. a protease sensitive cleavage site, an affinity tag such as the His-tag or the Strep-tag, a signal peptide, a retention peptide, a targeting peptide, such as a membrane translocation peptide or an additional effector domains such as an antibody fragment for tumor targeting associated with an anti-tumor toxin or an enzyme for prodrug-activation, etc.

As used herein, the terms “charged polypeptide domain” or “charged domain” refer to regions of a polypeptide, such as a fusion protein, comprising a plurality of amino acids independently selected from negatively charged amino acids and a plurality of amino acids independently selected from positively charged amino acids such that the segment is substantially electronically neutral. In addition to the positively charged and negatively charged amino acids, a charged domain can comprise one or more types of additional amino acids, e.g., uncharged amino acids, such that the segment is substantially electronically neutral.

In some embodiments of the fusion proteins disclosed herein, the plurality of negatively charged amino acids in the charged domain is independently selected from the group consisting of aspartic acid, glutamic acid, and derivatives thereof. In certain embodiments, the plurality of positively charged amino acids is independently selected from the group consisting of lysine, histidine, arginine, and derivatives thereof.

In some embodiments, positively charged amino acids and negatively charged amino acids constitute from about 20% to about 95%, from about 30% to about 95%, from about 40% to about 95%, from about 50% to about 95%, from about 40% to about 90%, from about 50% to about 90%, from about 40% to about 80%, or from about 50% to about 70% of the total number of amino acids present in the charged domain. In some embodiments, the positively charged amino acids constitute from about 10% to about 48%, from about 15% to about 48%, from 20% to about 48%, from about 25% to about 48%, from about 20% to about 45%, from about 25% to about 45%, from about 20% to about 40%, or from about 25% to about 35% of the total number of amino acids present in the charged domain. In some embodiments, the negatively charged amino acids constitute from about 10% to about 48%, from about 15% to about 48%, from 20% to about 48%, from about 25% to about 48%, from about 20% to about 45%, from about 25% to about 45%, from about 20% to about 40%, or from about 25% to about 35% of the total number of amino acids present in the charged domain.

The charged domain typically comprises about 6 or more amino acids. In some embodiments, the charged domain comprises from about 6 to about 1000 amino acids, from about 20 to about 1000 amino acids, from about 30 to about 1000 amino acids, from about 50 to about 1000 amino acids, from about 80 to about 1000 amino acids, from about 80 to about 600 amino acids, or from about 50 to about 500 amino acids.

The charged domain of the fusion proteins disclosed herein comprise negatively charged amino acids and positively charged amino acids in substantially equal numbers. In some embodiments, the ratio of the number of negatively charged amino acids to the number of positively charged amino acids is from about 1:0.5 to about 1:2, from about 1:07 to about 1:1.4, from about 1:0.8 to about 1:1.25, or from about 1:0.9 to about 1:1.1. Thus, the charged domain is substantially electronically neutral. In some embodiments, the polypeptide is substantially electronically neutral at pH f about 7.4.

In some embodiments, the charged domain comprises a plurality of lysines and a plurality of negatively charged amino acids selected from the group consisting of glutamic acid and aspartic acid. In some embodiments, the charged domain comprises a plurality of histidines and a plurality of negatively charged amino acids selected from the group consisting of glutamic acid and aspartic acid.

In some embodiments of the fusion proteins disclosed herein, the plurality of additional amino acids in the charged domain is selected from the group consisting of serine, asparagine, glycine, and proline. In some embodiments, the plurality of additional amino acids is selected from the group consisting of serine, glycine, and proline. In some embodiments, the plurality of additional amino acids is selected from the group consisting of serine and glycine. The charged domains of the disclosure can comprise only one type of additional amino acid (e.g., proline), two different additional amino acids (e.g., proline and glycine), three different additional amino acids (e.g, serine, glycine, and proline). In some embodiments, the charged domains comprise one additional amino acid. In some embodiments, the polypeptides comprise two additional amino acids.

In some embodiments, the plurality of additional amino acids is two or more prolines. In some embodiments, the plurality of additional amino acids is two or more glycines. In some embodiments, the plurality of additional amino acids is two or more serines.

In some embodiments, the charged domain comprises a plurality of lysines, a plurality of glutamic acids, and a plurality of additional amino acids selected from the group consisting of serine, glycine, and proline.

In some embodiments, the charged domain comprises a plurality of lysines, a plurality of glutamic acids, and a plurality of additional amino acids selected from the group consisting of glycine and proline.

In some embodiments, the charged domain consists essentially of a plurality of negatively charged amino acids; a plurality of positively charged amino acids; and a plurality of additional amino acids independently selected from the group consisting of proline, serine, threonine, asparagine, glutamine, glycine, and derivatives thereof, and optionally an affinity tag, such a histidine tag which can be used for affinity purification of the polypeptide. In some embodiments, the charged domain consists essentially of a plurality of glutamic acids; a plurality of lysines; and a plurality of additional amino acids independently selected from the group consisting of proline and glycine, and optionally an affinity tag, such a histidine tag which can be used for affinity purification of the polypeptide.

The amino acids in the charged domain can be arranged in any manner or sequence, such as in a manner described above. In some embodiments, the charged domain is a random coil polypeptide.

The fusion proteins disclosed herein comprise one or more functional domains. In some embodiments, the functional domain is a functional polypeptide. The terms “functional protein,” and “functional peptide” can be used interchangeably. In certain embodiments, peptides range from about 5 to about 40000, about 5 to about 20000, about 5 to about 10000, about 5 to about 5000, about 5 to about 1000, about 5 to about 750, about 5 to about 500, about 5 to about 250, about 5 to about 100, about 5 to about 75, about 5 to about 50, about 5 to about 40, about 5 to about 30, about 5 to about 25, about 5 to about 20, about 5 to about 15, or about 5 to about 10 amino acids in size.

In some embodiments, a functional polypeptide is a protein or a peptide, including an enzyme, a cytokine, a hormone, a growth factor, an antigen, an antibody, a characteristic portion of an antibody, a clotting factor, a regulatory protein, a signaling protein, a transcription protein, and a receptor. These include (IL-1 α), IL-1 β, IL-2, IL-3, IL-4, IL-5, IL-6, IL-11, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-31, IL-32, IL-33, colony stimulating factor-1 (CSF-1), macrophage colony stimulating factor, glucocerobrosidase, thyrotropin, stem cell factor, granulocyte macrophage colony stimulating factor, granulocyte colony stimulating factor (G-CSF), GM-CSF, (EOS)-CSF, CSF-1, EPO, organophosphorus hydrolase (OPH), interferon-alpha (IFN-α), consensus interferon-beta (IFN-β), interferon-gamma (IFN-γ), thrombopoietin (TPO), Cas9, Cas12a, Cas12b, Cas12c, Cas13a1, Cas13a2, Cas13b, Angiopoietin-1 (Ang-1), Ang-2, Ang-4, Ang-Y, angiopoietin-like polypeptide 1 (ANGPTL1), angiopoietin-like polypeptide 2 (ANGPTL2), angiopoietin-like polypeptide 3 (ANGPTL3), angiopoietin-like polypeptide 4 (ANGPTL4), angiopoietin-like polypeptide 5 (ANGPTL5), angiopoietin-like polypeptide 6 (ANGPTL6), angiopoietin-like polypeptide 7 (ANGPTL7), vitronectin, vascular endothelial growth factor (VEGF), angiogenin, activin A, activin B, activin C, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, bone morphogenic protein receptor II, brain derived neurotrophic factor, cardiotrophin-1, ciliary neutrophic factor, ciliary neutrophic factor receptor, cripto, cryptic, cytokine-induced neutrophil chemotactic factor 1, cytokine-induced neutrophil, chemotactic factor 2α, hepatitis B vaccine, hepatitis C vaccine, drotrecogin .alpha., cytokine-induced neutrophil chemotactic factor 2β, SLF, SCF, mast cell growth factor, endothelial cell growth factor, endothelin 1, epidermal growth factor (EGF), epigen, epiregulin, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor 11, fibroblast growth factor 12, fibroblast growth factor 13, fibroblast growth factor 16, fibroblast growth factor 17, fibroblast growth factor 19, fibroblast growth factor 20, fibroblast growth factor 21, fibroblast growth factor acidic, fibroblast growth factor basic, EPA, Lactoferrin, H-subunit ferritin, prostaglandin (PG) E1 and E2, glial cell line-derived neutrophic factor receptor .alpha.1, glial cell line-derived neutrophic factor receptor, growth related protein, growth related protein a, IgG, IgE, IgM, IgA, and IgD, α-galactosidase, β-galactosidase, DNAse, fetuin, leutinizing hormone, alteplase, estrogen, insulin, albumin, lipoproteins, fetoprotein, transferrin, thrombopoietin, urokinase, integrin, thrombin, Factor IX (FIX), Factor VIII (FVIII), Factor Vila (FVIIa), Von Willebrand Factor (VWF), Factor FV (FV), Factor X (FX), Factor XI (FXI), Factor XII (FXII), Factor XIII (FXIII), thrombin (FII), protein C, protein S, tPA, PAI-1, tissue factor (TF), ADAMTS 13 protease, growth related protein .beta., growth related protein, heparin binding epidermal growth factor, hepatocyte growth factor, hepatocyte growth factor receptor, hepatoma-derived growth factor, insulin-like growth factor I, insulin-like growth factor receptor, insulin-like growth factor II, insulin-like growth factor binding protein, keratinocyte growth factor, leukemia inhibitory factor, somatropin, antihemophiliac factor, pegaspargase, orthoclone OKT 3, adenosine deaminase, alglucerase, imiglucerase, leukemia inhibitory factor receptor .alpha., nerve growth factor nerve growth factor receptor, neuropoietin, neurotrophin-3, neurotrophin-4, oncostatin M (OSM), placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor .alpha., platelet derived growth factor receptor .beta., pre-B cell growth stimulating factor, stem cell factor (SCF), stem cell factor receptor, TNF, TNF0, TNF1, TNF2, transforming growth factor α, hymic stromal lymphopoietin (TSLP), tumor necrosis factor receptor type I, tumor necrosis factor receptor type II, urokinase-type plasminogen activator receptor, phospholipase-activating protein (PUP), insulin, lectin ricin, prolactin, chorionic gonadotropin, follicle-stimulating hormone, thyroid-stimulating hormone, tissue plasminogen activator (tPA), leptin, Enbrel (etanercept), activin, inhibin, leukemic inhibitory factor, oncostatin M, MIP-1-C, MIP-1 B; MIP-2-C, GRO-C; MIP-2-B and platelet factor-4.

In some embodiments, the functional domain can comprise a designed functional polypeptide sequence. In some embodiments, the functional polypeptide sequence is a domain or fragment of a functional polypeptide. In some embodiments, the functional polypeptide sequence is a recognition sequence, which optionally results in stoichiometric binding or modification of the polypeptide. In some embodiments, the functional polypeptide sequence is a sequence useful for promoting expression or purification of the fusion polypeptide. In some embodiments, the functional polypeptide sequence is a structural motif of a secondary or higher nature, comprising helices, sheets, turns, folds, and super domains. In some embodiments, the functional polypeptide sequence is a linker sequence that exists between two other domains.

In some embodiments, the functional polypeptide domains can be modified through rational design, directed evolution, or another technique yielding a functional protein improved in at least one aspect of performance.

The domains of the fusion proteins disclosed herein can contain L-amino acids, D-amino acids, or a combination thereof, and may contain any of a variety of amino acid modifications or analogs known in the art. In one embodiment, useful modifications comprise terminal acetylation, amidation, site-specific conversion of cysteine to formylglycine. In some embodiments, the functional domain and the protective domain may comprise natural amino acids, unnatural amino acids, synthetic amino acids, and combinations thereof, as described herein.

In some embodiments, the charged domain acts as a protective domain, i.e., a domain that provides advantageous properties to a molecule to which it is attached, such as enhanced stability, improved solubility, and/or improved pharmacokinetic properties. The terms “protective domain”, “protective polypeptide domain”, as well as “mixed charge protective polypeptide domain” can be used interchangeably.

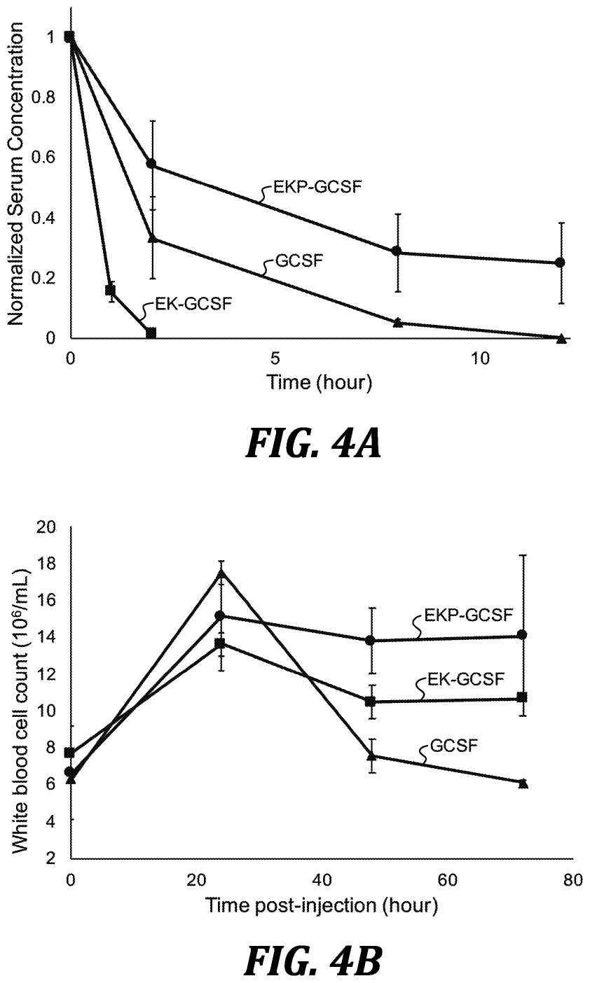

The fusion proteins disclosed herein have advantageous properties compared to the comparable proteins that do not comprise the one or more charged domains as disclosed herein. As illustrated in the examples below and in FIG. 4A, an exemplary fusion protein EKP-GCSF comprising a granulocyte colony-stimulating factor protein functional domain (GCSF, SEQ ID NO: 10) and an exemplary charged polypeptide domain comprising amino acids glutamic acid (E), lysine (K), and proline (P) (EKP) showed enhanced circulation profile when compared to the GCSF protein alone. Surprisingly, the EKP-GCSF (SEQ ID NO: 2) demonstrated enhanced circulation profile compared to a fusion protein EK-GCSF (SEQ ID NO: 8), which contained a charged domain comprising only glutamic acid (E) and lysine (K). The exemplary fusion protein EKP-GCSF also exhibited increased activity/efficacy when compared to EK-GCSF or GCSF alone as determined through a white blood cell counts assay and illustrated in FIG. 4B.

Additionally, as demonstrated in FIG. 6, an exemplary fusion protein (EKP-IFNα2a, SEQ ID NO: 14) comprising an exemplary EKP polypeptide domain fused to a terminus of Interferon alpha 2a (IFNα2a), demonstrated a more favorable pharmacokinetic profile as compared to the IFNα2a protein itself (IFNα2a, SEQ ID NO: 16) or an IFNα2a fusion protein with a charged domain comprising only glutamic acid (E) and lysine (K) (EK-IFNα2a, SEQ ID NO: 12).

### Preparation of Polypeptides and Fusion Proteins

The fusion proteins and polypeptides disclosed herein can be prepared in any suitable manner, for example, using molecular cloning techniques.

Accordingly, in an aspect, the disclosure provides a nucleic acid comprising a sequence encoding a fusion protein or a polypeptide disclosed herein. In one embodiment, the present invention provides isolated nucleic acids encoding the polypeptide, e.g., a fusion protein, of any aspect of the invention. The isolated nucleic acid sequence can comprise RNA or DNA. As used herein, “isolated nucleic acids” are nucleic acids that have been removed from their normal surrounding nucleic acid sequences in the genome or in cDNA sequences. Such isolated nucleic acid sequences can further comprise additional sequences useful for promoting expression and/or purification of the encoded polypeptide as previously mentioned.

The nucleic acid encoding a fusion protein of the disclosure or a polypeptide of the disclosure can be incorporated into a suitable expression vector. An expression vector or an expression construct is a DNA molecule that carries a specific gene into a host cell and uses the cell's protein synthesis machinery to produce the protein encoded by the gene. An expression vector also contains elements essential for gene expression, such as a promoter region operatively linked to the gene, which allows efficient transcription of the gene. The expression of the protein can be controlled, and the protein is only produced in significant quantity when necessary, by using an inducer. E. coli is commonly used as the host for protein production, but other cell types can also be used, such as yeast, insect cells, and mammalian cells.

Thus, in an aspect, provided herein is a cell comprising the nucleic acid encoding a fusion protein or a polypeptide of the disclosure. The cell can be a prokaryotic cell or eukaryotic cell.

In some embodiments, a polypeptide or a fusion protein disclosed herein can be synthesized using any suitable expression system, such as the Escherichia coli expression system, Bacillus subtilis expression system, or any other prokaryotic expression system.

In one embodiment, a polypeptide or a fusion protein disclosed herein can be synthesized using the Pichia pastoris expression system. In another embodiment, a polypeptide or a fusion protein disclosed herein can be synthesized using the Human Embryonic Kidney 293 expression system. In another embodiment, a polypeptide or a fusion protein disclosed herein can be synthesized using the Chinese Hamster Ovary expression system. In one embodiment, a polypeptide or a fusion protein disclosed herein can be synthesized using a prokaryotic or eukaryotic cell free expression system.

Recovery and purification of the polypeptides and fusion proteins disclosed herein can be achieved by any method or a combination of such methods. In some embodiments, protein precipitation techniques can be used. In some embodiments, a polypeptide or a fusion protein disclosed herein can be purified using size exclusion chromatography. In some embodiments, a polypeptide or a fusion protein disclosed herein can be purified using ion exchange chromatography. In some embodiments, a polypeptide or a fusion protein disclosed herein can be purified using desalting columns. In some embodiments, a polypeptide or a fusion protein disclosed herein can be purified using affinity chromatography. In some embodiments, a polypeptide or a fusion protein disclosed herein can be purified using hydrophobic or hydrophilic properties. In some embodiments, a polypeptide or a fusion protein disclosed herein can be purified using matrix-free electrophoresis techniques.

While exemplary embodiments have been illustrated and described, it will be appreciated that various changes can be made therein without departing from the spirit and scope of the invention. While each of the elements of the present invention is described herein as containing multiple embodiments, it should be understood that, unless indicated otherwise, each of the embodiments of a given element of the present invention is capable of being used with each of the embodiments of the other elements of the present invention and each such use is intended to form a distinct embodiment of the present invention.

As can be appreciated from the disclosure above, the present invention has a wide variety of applications. The invention is further illustrated by the following examples, which are provided for the purpose of illustrating, not limiting, the invention.

### EXAMPLES

### Example 1: Preparation and Characterization of a Series of Polypeptides Fused to Terminus of Granulocyte Colony-Stimulating Factor (GCSF)

In this example, DNA sequences (SEQ ID NOS: 1, 3, and 5) encoding proteins comprising a domain comprising the amino acids E and K as well X (domain denoted as EKX), where X in this example is G (domain denoted as EKG, amino acids 2-292 of SEQ ID NO: 4), P (domain denoted as EKP, amino acids 2-272 of SEQ ID NO: 2), or a mixture of G and P (domain denoted as EKPG, amino acids 2-278 of SEQ ID NO: 6), fused to the N-terminus of granulocyte colony-stimulating factor (GCSF), with an additional 6×His tag fused to the C-terminus of GCSF (e.g., EKX-GCSF-His) were cloned into the pMAL-c5E expression vector. The pMAL-c5E vector contained a DNA sequence encoding maltose binding protein (MBP) with an enterokinase cleavage site. EKX-GCSF were cloned such that MBP with the enterokinase site is on the N-terminal of the EKX-GCSF. MBP has been shown to enhance the expression and solubility of GCSF fusion proteins, which can be cleaved off using enterokinase at its target cleavage site leaving only the desired EKX-GCSF fusion protein. The pMAL-c5E-EKX-GCSF-His constructed was transformed into BL21 (DE3) E. coli competent cells. Transformed E. coli were grown in Terrific Broth (TB) with 100 μg/mL of ampicillin at 37° C. to an optical density (OD600) of 0.5 at which point the expression was induced with 1 mM isopropyl β-D-1-thiogalactopyranoside (IPTG). At this point, the temperature was shifted to 30° C. and grown for 6 hours. The culture was harvested by pelleting cells. Pellets were resuspended in 20 mM sodium phosphate, 6 M GnHcl, 500 mM NaCl, 10 mM imidazole, pH 8 and lysed with freeze-thaws and sonication. Cell debris were then pelleted with the protein of desire left in the supernatant. Lipids were removed via ethanol precipitation and the protein was resuspended in the original buffer. The resulting sample was loaded onto a Nuvia IMAC column (BioRad). The protein was eluted using the same buffer at pH 4.

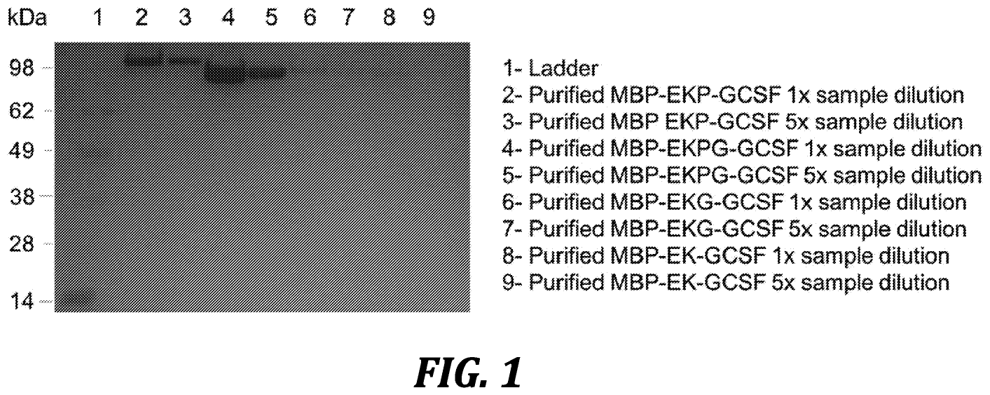

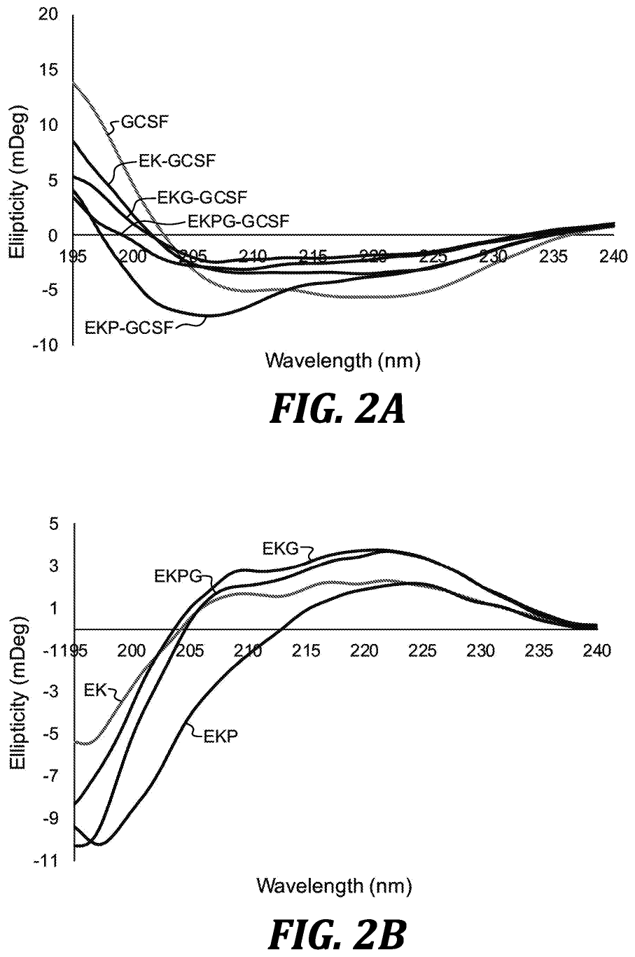

This resulting protein was precipitated in ethanol to get rid of guanidine hydrochloride and resuspended in SDS-PAGE loading buffer for western blot. Protein of interest was transferred on to a polyvinylidene difluoride (PVDF) membrane probed with monoclonal anti-hGCSF antibody (Invitrogen) for detecting (FIG. 1). The bands appeared with indicating the success of production of protein of interest. MBP attached to the fusion protein from expression was cleaved using enterokinase at 20° C. for 16 hr. The final products after MBP cleavage were analyzed utilizing circular dichroism to determine the structure of the fusion protein. Equimolar amounts 50 μg/mL of the resulting proteins EKP-GCSF (SEQ ID NO: 2, 50 μg/mL), EKPG-GCSF (SEQ ID NO: 6, 50 μg/mL), EKG-GCSF (SEQ ID NO: 4, 50 μg/mL), EK-GCSF (V SEQ ID NO: 8, 50 μg/mL) and GCSF alone (SEQ ID NO: 10, 20 μg/mL) were analyzed using Jasco 720 circular dichroism instrument in 10 mM potassium phosphate buffer pH 8 (FIG. 2A). To determine the structure of polypeptides themselves, EKP, EKPG, EKG, and EK, the GCSF profile was subtracted from that of the fusion protein variants (FIG. 2B). The profiles indicated the presence of random coil with increased random coil in EKP, EKPG, and EKG compared to EK.

### Example 2: The Pharmacokinetics and Pharmacodynamics Properties of a Series of Polypeptides Fused to Terminus of GCSF

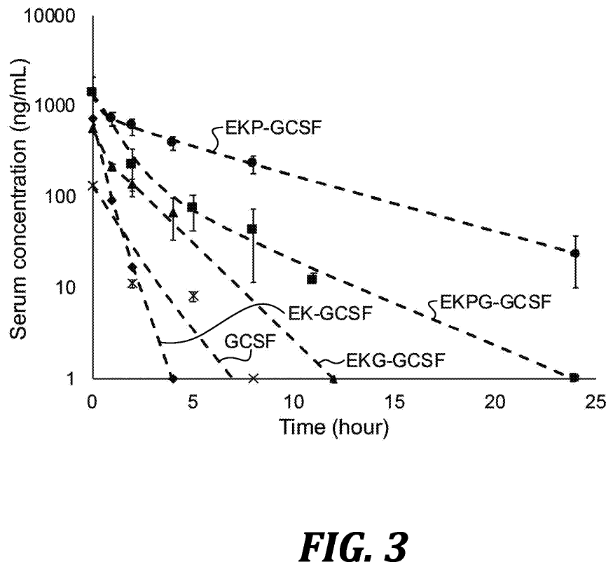

The pharmacokinetics profiles of the fusion protein variants obtained as described above were determined in vivo using C57BL/6 Mice (6 weeks old) by retro-orbital injection for EKP-GCSF (SEQ ID NO: 2), EKPG-GCSF (SEQ ID NO: 6), EKG-GCSF (SEQ ID NO: 4), EK-GCSF (SEQ ID NO: 8), and GCSF (SEQ ID NO: 10), (20 nmol/kg) alone at t=0 hr. Blood was drawn at the indicated time points from the chins of the mice. Serum concentrations were determined using a capture ELISA assay using anti-hGCSF monoclonal antibody (3316-Invitrogen) and anti-hGCSF polyclonal antibody (R&D systems) (FIG. 3). Standard curves were developed for each variant (EKP-GCSF, EKPG-GCSF, EKG-GCSF, EK-GCSF, and GCSF) to account for differential binding of antibodies to GCSF epitopes.

To further elucidate the pharmacokinetic and pharmacodynamics properties of these variants, EK-GCSF, EKP-GCSF, and GCSF (10 nmol/kg) were injected into Sprague-Dawley rats by tail vein injection. Blood was drawn at indicated time points post injection via tail vein blood draw. Serum concentrations were determined using a capture ELISA assay using anti-hGCSF monoclonal antibody (3316-Invitrogen) and anti-hGCSF polyclonal antibody (R&D systems). Standard curves were developed for each variant (EKP-GCSF, EK-GCSF, and GCSF) to account for differential binding of antibodies to GCSF epitopes. Serum concentrations normalized to initial serum concentrations at t=0 hr (FIG. 4A). EKP-GCSF showed enhanced circulation profile when compared to EK-GCSF or GCSF alone. The efficacy of the fusion protein variant was determined through white blood cell counts (WBC). The WBC were determined at indicated time points by Medix LeukoTic Bluplus WBC test kit according to the manufacturer's instructions (FIG. 4B). EKP-GCSF also exhibited increased activity/efficacy when compared to EK-GCSF or GCSF alone as the white blood cell counts for animals injected with EKP-GCSF had a higher and longer elevation.

### Example 3: Preparation, Characterization, and Pharmacokinetic Profile of a Series of Polypeptides Fused to Terminus of Interferon Alpha 2a (IFNα2a)

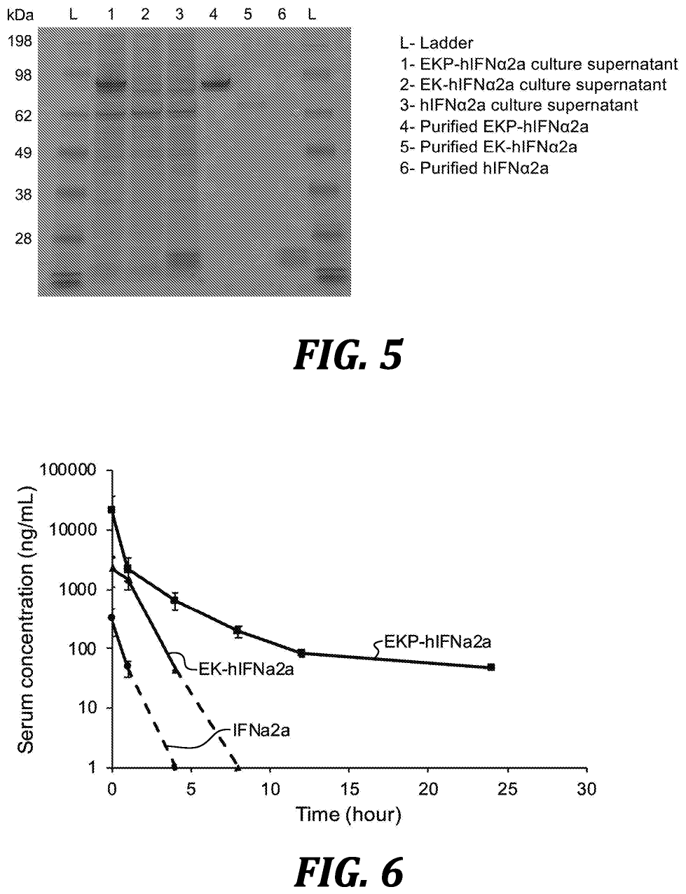

In this example, DNA sequences encoding a domain comprising the amino acids E and K with or without P were fused to the N-terminal of hIFNα2a, yielding EK-hIFNα2a and EKP-hIFNα2a fusion proteins. A HA-tag (YPYDVPDYA) was added to the N-terminus of the fusion protein for the detection of full-length products. For efficient extracellular secretion in mammalian cells, the innate secretion signal sequence hIFNα2a was deleted and replaced with the human tissue plasminogen activator (tPA) leader sequence. The proteins EK-hIFNα2a (SEQ ID NO: 12), EKP-hIFNα2a (SEQ ID NO: 14), and hIFNα2a (SEQ ID NO: 16), encoded by these resulting DNA SEQ ID NO: 11, SEQ ID NO: 13, and SEQ ID NO: 15, respectively, were prepared as follows. The expression cassette was cloned into the pcDNA3.1+ mammalian cell expression vector containing a CMV promoter. The FreeStyle™ 293-F cell (HEK293-F, ThermoFisher, USA), derived from HEK293 cell line, was used for protein expression. Cells were first seeded at a density of 10⁶ cells/mL in 30 mL F17 medium and incubated at 37° C. in a humidified atmosphere of 5% CO₂ on an orbital shaker platform rotating at 120 rpm. Then, the constructed plasmid was complexed with polyethylenimine (PEI) at a N/P ratio of 3:1 and incubated with HEK293-F. After 72 hours, the culture supernatants were collected and protein were purified by HA-tag specific antibodies using Pierce™ Anti-HA Agarose (ThermoFisher, US). SDS-PAGE analysis confirmed the success of extracellular expression of hIFNα2a, EK-hIFNα2a and EKP-hIFNα2a in HEK293-F after transfection of plasmids, respectively (FIG. 5). Bands around 20 kDa were detected which agrees with the size of hIFNα2a (19.2 kDa). The band of EK-hIFNα2a and EKP-hIFNα2a (both 49.2 kDa) were also detected. EKP-hIFNα2a exhibited significant retarded migration on SDS-PAGE may due to the nature of random coil structure of the EKP polypeptide.

The pharmacokinetics profiles of the hIFNα2a fusion protein variants were determined through in vivo testing in C57BL/6 mice (6 weeks old). Three mice in each experimental group were administered with 50 nmol/kg EKP-hIFNα2, EK-hIFNα2a, and hIFNα2a via retro-orbital injection at t=0 hr. After administration, blood samples were collected from chin bleeds of each animal at 0, 1, 4, 8, 12, 24, 48 hours post-injection. Serum concentrations of proteins from each sample were quantified by a capture ELISA using anti-HA tag antibody (NB600-363, Novus) and anti-human interferon alpha 2 polyclonal antibody (MBS2527079, MyBioSource) (FIG. 6). Standard curves were developed for each variant (HA-EKP-hIFNα2a, HA-EK-hIFNα2a, and HA-hIFNα2a) to account for differential binding of antibodies to HA and hIFNα2a epitopes.

### Example 4: Production of a Series of Polypeptides Fused to Enhanced Green Fluorescent Protein (eGFP)

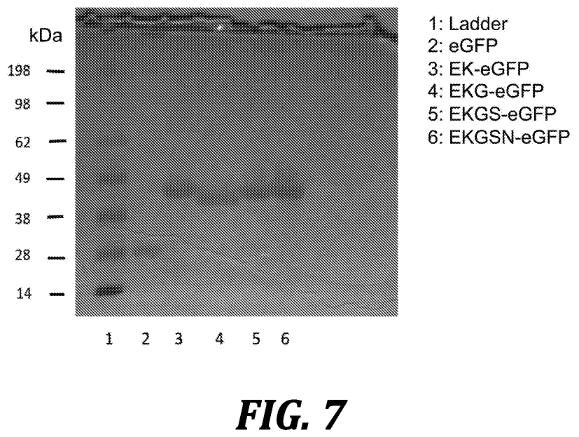

In this example, DNA (SEQ ID NO: 17, 19, 21, and 23) encoding proteins comprising 10 kDa segments of EK (amino acids 249-330 of SEQ ID NO: 18), EKGSN (amino acids 246-346 of SEQ ID NO: 20), EKG (amino acids 247-342 of SEQ ID NO: 22), and EKGS (amino acids 247-346 of SEQ ID NO: 24) fused to the C-terminal of eGFP (all proteins denoted as EKX-eGFP) were synthesized and cloned into pET20b+ plasmids for expression into the cytoplasm. BL21 (DE3) E. coli were transformed with EKX-eGFP plasmids. Transformed E. coli were grown in Terrific Broth (TB) with 100 μg/mL of ampicillin at 37° C. to an optical density (OD600) of 0.5 at which point the expression was induced with 1 mM isopropyl β-D-1-thiogalactopyranoside (IPTG). At this point, the temperature was shifted to 30° C. and grown for 6 hours. The culture was harvested by centrifuging the culture at 10000 rpm for 10 minutes to pellet the cells. Cell pellets were resuspended in phosphate buffered saline (PBS) and sonicated with a probe sonicator to lyse the cell Ammonium sulfate was added to 2M and any precipitated protein was removed by centrifugation. The supernatant was applied to a phenyl hydrophobic interaction chromatography column (HIC) and eluted with a gradient of decreasing ammonium sulfate concentration. Fractions containing eGFP (and polypeptide variants) were pooled and applied to a size exclusion chromatography column (SEC) equilibrated with PBS. Fractions were containing eGFP were pooled again and applied to an anion exchange column (AEX) column. Protein was eluted using an increasing sodium chloride gradient (up to 1M). Fractions containing eGFP were pooled and analyzed on SDS-PAGE (FIG. 7). Yields were calculated using bicinchronic acid (BCA) assay and reported for each 1-liter batch (Table 1).

TABLE 1
Purification yield from 1-liter shaker flask
expression of eGFP and EKX-eGFP proteins.

| | Yield |
|---|---|
| Variant | (mg/L of Culture) |
| eGFP (SEQ ID NO: 26) | 9.1 |
| EK-eGFP (SEQ ID NO: 18) | 2.9 |
| EKG-eGFP (SEQ ID NO: 22) | 0.84 |
| EKGS-eGFP (SEQ ID NO: 24) | 29 |
| EKGSN-eGFP (SEQ ID NO: 20) | 16 |

1. A polypeptide comprising:
a) a plurality of negatively charged amino acids;
b) a plurality of positively charged amino acids; and
c) a plurality of additional amino acids independently selected from the group consisting of proline, serine, threonine, asparagine, glutamine, glycine, and derivatives thereof; and
wherein the ratio of the number of positively charged amino acids to the number of positively charged amino acids is from about 1:0.5 to about 1:2.

2. The polypeptide of claim 1, wherein the plurality of negatively charged amino acids is independently selected from the group consisting of aspartic acid, glutamic acid, and derivatives thereof.

3. The polypeptide of claim 1, wherein the plurality of positively charged amino acids is independently selected from the group consisting of lysine, histidine, arginine, and derivatives thereof.

4. The polypeptide of claim 1, wherein the positively charged amino acids and negatively charged amino acids constitute from about 20% to about 95% of the total number of amino acids present in the charged domain.

5. The polypeptide of claim 1, wherein the polypeptide comprises from about 6 to about 1000 amino acids.

6. The polypeptide of claim 1, wherein the ratio of positively charged amino acids to negatively charged amino acids is from about 1:07 to about 1:1.4.

7. The polypeptide of claim 1, wherein the polypeptide comprises at least two pairs comprising a positively charged amino acid adjacent to a negatively charged amino acid.

8-9. (canceled)

8. The polypeptide of claim 1, wherein the polypeptide consists essentially of:
a) a plurality of negatively charged amino acids;
b) a plurality of positively charged amino acids; and
c) a plurality of additional amino acids independently selected from the group consisting of proline, serine, threonine, asparagine, glutamine, glycine, and derivatives thereof.

9. The polypeptide of claim 1, wherein the polypeptide comprises a plurality of lysines and a plurality of negatively charged amino acids selected from the group consisting of glutamic acid and aspartic acid.

12. (canceled)

10. The polypeptide of claim 11, wherein the plurality of additional amino acids is selected from the group consisting of serine, asparagine, glycine, and proline.

14. (canceled)

11. The polypeptide of claim 11, wherein the plurality of additional amino acids is selected from the group consisting of serine and glycine.

16-21. (canceled)

12. A bioconjugate comprising at least one polypeptide of claim 1 covalently coupled to a biomolecule.

23. (canceled)

13. A fusion protein comprising one or more functional domains linked to one or more charged domains, wherein the one or more charged domains comprises:
a) a plurality of negatively charged amino acids;
b) a plurality of positively charged amino acids; and
c) a plurality of additional amino acids independently selected from the group consisting of proline, serine, threonine, asparagine, glutamine, glycine, and derivatives thereof; and
wherein the ratio of the number of positively charged amino acids to the number of positively charged amino acids is from about 1:0.5 to about 1:2.

14. The fusion protein of claim 24, wherein the plurality of negatively charged amino acids is independently selected from the group consisting of aspartic acid, glutamic acid, and derivatives thereof.

15. The fusion protein of claim 24, wherein the plurality of positively charged amino acids is independently selected from the group consisting of lysine, histidine, arginine, and derivatives thereof.

16. The fusion protein of claim 24, wherein the positively charged amino acids and negatively charged amino acids constitute from about 20% to about 95% of the total number of amino acids present in the charged domain.

28-32. (canceled)

17. The fusion protein of claim 24, wherein the one or more charged domains consists essentially of:
a) a plurality of negatively charged amino acids or latent negatively charged amino acids;
b) a plurality of positively charged amino acids or latent positively charged amino acids; and
c) a plurality of additional amino acids independently selected from the group consisting of proline, serine, threonine, asparagine, glutamine, glycine, and derivatives thereof.

34-44. (canceled)

18. A nucleic acid comprising a sequence encoding the fusion protein of claim 24.

19. An expression vector comprising the nucleic acid of claim 45.

20. A cell comprising the nucleic acid of claim 45.

48-51. (canceled)


SEQUENCE LISTING (26 sequences)

SEQ ID NO: 1 - DNA, 1344 bp, Artificial sequence
  Synthetic
atggagaagccgaaagagccggaaaagccggagaagccgaaagaaccgaaggaaccggaaaaaccgaaggagccggagaaaccggaaaaaccgaaagaaccgaaggagccggaaaaaccgaaagagccggagaaaccggagaaaccgaaggaaccgaaagaaccggagaaaccgaaagaaccggaaaaaccggaaaagccgaaagaaccgaaagagcctgagaaaccgaaagagccggaaaaaccggagaagccgaaggagccgaaggaacctgagaagcctaaggagccggagaagcctgaaaaacctaaggagcctaaggaacctgagaagcccaaggaaccggagaaacctgaaaagccgaaagagccgaaggaacccgagaaacctaaagaaccggagaagccggaaaagccgaaggagccgaaagaacctgagaagcctaaagaacctgagaagcccgagaaaccgaaggagccgaaggagccggagaagccgaaggaaccggagaaacccgaaaaaccgaaggagcctaaagaacccgagaaacccaaggaaccggagaagccggagaaacctaaggagccgaaagaacccgagaaaccaaaggaaccggaaaagcctgaaaaacccaaggagcctaaagaaccggaaaagccgaaggaaccggaaaagcccgaaaaacctaaggaacctaaggaacccgagaagcctaaggaaccggaaaagccagaaaaacctaaggaacccaaggaacccgagaagcccaaggagccggaaaagccggaaaagcctaaggaaccgaaggaaccgaagcttatgaccccgctgggtccggcgagcagcctgccgcagagcttcctgctgaaatgcctggaacaagtgcgtaagatccaaggtgacggcgcggcgctgcaagagaaactgtgcgcgacctacaagctgtgccacccggaggaactggttctgctgggtcacagcctgggtattccgtgggcgccgctgagcagctgcccgagccaggcgctgcaactggcgggttgcctgagccagctgcacagcggtctgttcctgtatcagggcctgctgcaagcgctggaaggtatcagcccggagctgggtccgaccctggataccctgcaactggacgtggcggattttgcgaccaccatttggcagcaaatggaagaactgggtatggcgccggcgctgcagccgacccaaggtgcgatgccggcgtttgcgagcgcgtttcaacgtcgtgcgggtggcgtgctggttgcgagccacctgcagagcttcctggaagtgagctaccgtgttctgcgtcacctggcgcagccg

SEQ ID NO: 2 - protein, 448 aa, Artificial sequence
  Synthetic
MEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPKLMTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 3 - DNA, 1407 bp, Artificial sequence
  Synthetic
atggagaagggtaaagagggcgaaaagggcgagaagggcaaagaaggtaaagagggcgagaaaggcaaagagggcgagaagggcgaaaaaggtaaagaaggtaaagagggcgaaaaaggcaaagagggcgaaaagggcgaaaagggtaaagaaggtaaagagggcgaaaaggggaaagagggcgaaaagggcgagaaaggtaaagaaggtaaagagggcgaaaagggtaaagagggcgagaagggcgagaaaggaaaagaaggtaaagagggcgaaaagggcaaagagggcgaaaagggcgagaaggggaaagaaggtaaagagggcgaaaagggcaaagagggcgaaaagggcgagaagggtaaagaaggtaaagagggcgaaaagggaaaagagggcgaaaagggcgagaagggaaaagaaggtaaagagggcgaaaagggcaaagagggcgaaaagggcgagaaggggaaagaaggtaaagagggcgaaaaggggaaagagggcgaaaagggcgagaaggggaaagaaggtaaagagggcgaaaaggggaaagagggcgaaaagggcgagaaggggaaagaaggtaaagagggcgaaaaggggaaagagggcgaaaagggcgagaaggggaaagaaggtaaagagggcgaaaaggggaaagagggcgaaaagggcgagaaggggaaagaaggtaaagagggcgaaaaggggaaagagggcgaaaagggcgagaaggggaaagaaggtaaagagggcgaaaaggggaaagagggcgaaaagggcgagaaggggaaagaaggtaaagagggtgaaaagggcaaagagggtgagaaaggcgaaaaaggtaaagagggtaaagagggtgaaaaaggtaagcttatgaccccgctgggtccggcgagcagcctgccgcagagcttcctgctgaaatgcctggaacaagtgcgtaagatccaaggtgacggcgcggcgctgcaagagaaactgtgcgcgacctacaagctgtgccacccggaggaactggttctgctgggtcacagcctgggtattccgtgggcgccgctgagcagctgcccgagccaggcgctgcaactggcgggttgcctgagccagctgcacagcggtctgttcctgtatcagggcctgctgcaagcgctggaaggtatcagcccggagctgggtccgaccctggataccctgcaactggacgtggcggattttgcgaccaccatttggcagcaaatggaagaactgggtatggcgccggcgctgcagccgacccaaggtgcgatgccggcgtttgcgagcgcgtttcaacgtcgtgcgggtggcgtgctggttgcgagccacctgcagagcttcctggaagtgagctaccgtgttctgcgtcacctggcgcagccg

SEQ ID NO: 4 - protein, 469 aa, Artificial sequence
  Synthetic
MEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKEGEKGEKGKEGKEGEKGKLMTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 5 - DNA, 1362 bp, Artificial sequence
  Synthetic
atggagaagccgaaagagggtgaaaagccggagaagggtaaagaaggcaaagagccggaaaaaccgaaagagggtgagaagccggagaagggcaaagaaccgaaagagggtgaaaaaccgaaagagggcgagaaaccggaaaaaggcaaagaaggcaaggagccggaaaagccgaaagagggtgagaaaccggaaaagggtaaggagcctaaagagggtgaaaaacctaaagagggcgagaagccggaaaaaggtaaagaaggcaaggaaccggagaaacctaaagagggtgagaaacctgaaaaaggtaaggagcccaaagagggtgaaaaacccaaagagggcgaaaaaccggaaaagggcaaagaaggcaaggaacctgagaaacccaaagagggtgagaaacccgaaaaaggtaaagagcctaaagagggtgagaagcctaaagagggcgaaaagcctgaaaaaggcaaagaaggcaaagaaccggagaaaccaaaagagggtgagaaaccagaaaaaggtaaagagcccaaagagggtgagaagcccaaagagggcgaaaagcccgaaaaaggcaaagaaggcaaagagcctgagaaaccgaaagagggtgaaaagccagaaaaaggtaaagaacctaaagagggtgaaaagcctaaagagggcgaaaagccagaaaagggtaaagaaggcaaggagcctgagaaaccgaaagagggtgaaaagcccgaaaagggtaaagagccaaaagagggtgaaaaaccaaaagagggtgaaaagccagagaaaggcaaagaaggcaaagagccagaaaagcctaaagagggtaagcttatgaccccgctgggtccggcgagcagcctgccgcagagcttcctgctgaaatgcctggaacaagtgcgtaagatccaaggtgacggcgcggcgctgcaagagaaactgtgcgcgacctacaagctgtgccacccggaggaactggttctgctgggtcacagcctgggtattccgtgggcgccgctgagcagctgcccgagccaggcgctgcaactggcgggttgcctgagccagctgcacagcggtctgttcctgtatcagggcctgctgcaagcgctggaaggtatcagcccggagctgggtccgaccctggataccctgcaactggacgtggcggattttgcgaccaccatttggcagcaaatggaagaactgggtatggcgccggcgctgcagccgacccaaggtgcgatgccggcgtttgcgagcgcgtttcaacgtcgtgcgggtggcgtgctggttgcgagccacctgcagagcttcctggaagtgagctaccgtgttctgcgtcacctggcgcagccg

SEQ ID NO: 6 - protein, 454 aa, Artificial sequence
  Synthetic
MEKPKEGEKPEKGKEGKEPEKPKEGEKPEKGKEPKEGEKPKEGEKPEKGKEGKEPEKPKEGEKPEKGKEPKEGEKPKEGEKPEKGKEGKEPEKPKEGEKPEKGKEPKEGEKPKEGEKPEKGKEGKEPEKPKEGEKPEKGKEPKEGEKPKEGEKPEKGKEGKEPEKPKEGEKPEKGKEPKEGEKPKEGEKPEKGKEGKEPEKPKEGEKPEKGKEPKEGEKPKEGEKPEKGKEGKEPEKPKEGEKPEKGKEPKEGEKPKEGEKPEKGKEGKEPEKPKEGKLMTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 7 - DNA, 1251 bp, Artificial sequence
  Synthetic
atggagaaggagaaggagaaggaaaaggagaaggagaaagagaaggagaaggagaaagaaaaggagaaggaaaaggaaaaggagaaggaaaaagagaaggagaaggaaaaagaaaaggagaaagagaaggaaaaggagaaagagaaagagaaggagaaagagaaagaaaaggagaaagaaaaggacaaggagaaagaaaaagagaaggagaaagaaaaagaaaaggagaaagaaaaagaaaaagagaaggaaaaggaaaaagagaaggaaaaagagaaagagaaggaaaaagaaaaagagaaggaaaaagaaaaggaaaaggaaaaggaaaaagaaaaagaaaaggaaaaagagaaagagaaagacaaagaaaaagagaaagaaaaggaaaaagaaaaagaaaaggaaaaagaaaaagagaaagaaaaagaaaaggagaaagagaaagaaaaggaaaaggaaaaagaaaaggagaaggagaaggagaaagaaaaagagaaggagaaagaaaaggaaaaggagaaagaaaaggagaaagagaaggacaaagagaaagaaaaggagaaggagaaggagaaggagaaggagaaggagaaggagaaggagaaggagaaggagaaggagaaggagaaggagaaggagaaggagaaggagaaggagaaggagaaagaaaaagaaaaagaaaaagaaaaagaaaaagaaaaagaaaaagaaaaagaaaagaagcttaccccgctgggtccggcgagcagcctgccgcagagcttcctgctgaaatgcctggaacaagtgcgtaagatccaaggtgacggcgcggcgctgcaagagaaactgtgcgcgacctacaagctgtgccacccggaggaactggttctgctgggtcacagcctgggtattccgtgggcgccgctgagcagctgcccgagccaggcgctgcaactggcgggttgcctgagccagctgcacagcggtctgttcctgtatcagggcctgctgcaagcgctggaaggtatcagcccggagctgggtccgaccctggataccctgcaactggacgtggcggattttgcgaccaccatttggcagcaaatggaagaactgggtatggcgccggcgctgcagccgacccaaggtgcgatgccggcgtttgcgagcgcgtttcaacgtcgtgcgggtggcgtgctggttgcgagccacctgcagagcttcctggaagtgagctaccgtgttctgcgtcacctggcgcagccg

SEQ ID NO: 8 - protein, 417 aa, Artificial sequence
  Synthetic
MEKEKEKEKEKEKEKEKEKEKEKEKEKEKEKEKEKEKEKEKEKEKEKEKEKEKEKEKEKEKDKEKEKEKEKEKEKEKEKEKEKEKEKEKEKEKEKEKEKEKEKEKEKEKEKEKEKEKEKEKDKEKEKEKEKEKEKEKEKEKEKEKEKEKEKEKEKEKEKEKEKEKEKEKEKEKEKEKEKEKDKEKEKEKEKEKEKEKEKEKEKEKEKEKEKEKEKEKEKEKEKEKEKEKEKEKEKEKEKEKKLTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 9 - DNA, 525 bp, Artificial sequence
  Synthetic
atgaccccgctgggtccggcgagcagcctgccgcagagcttcctgctgaaatgcctggaacaagtgcgtaagatccaaggtgacggcgcggcgctgcaagagaaactgtgcgcgacctacaagctgtgccacccggaggaactggttctgctgggtcacagcctgggtattccgtgggcgccgctgagcagctgcccgagccaggcgctgcaactggcgggttgcctgagccagctgcacagcggtctgttcctgtatcagggcctgctgcaagcgctggaaggtatcagcccggagctgggtccgaccctggataccctgcaactggacgtggcggattttgcgaccaccatttggcagcaaatggaagaactgggtatggcgccggcgctgcagccgacccaaggtgcgatgccggcgtttgcgagcgcgtttcaacgtcgtgcgggtggcgtgctggttgcgagccacctgcagagcttcctggaagtgagctaccgtgttctgcgtcacctggcgcagccg

SEQ ID NO: 10 - protein, 175 aa, Artificial sequence
  Synthetic
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 11 - DNA, 1299 bp, Artificial sequence
  Synthetic
atggacgccatgaagaggggcctgtgctgcgtgctgctgctgtgcggagccgtgttcgtgagcccctccgcctcttacccatacgatgttccagattacgctgagaaggaaaaagagaaggaaaaggaaaaggagaaagaaaaggagaaagagaaagagaaggaaaaggagaaagagaaggagaaggaaaaagaaaaggagaaggaaaaggagaaggaaaaggagaaggagaaggaaaaggaaaaagagaaggagaaggagaaggagaaggagaaggagaaggagaaggagaaggagaaggagaaggagaaggagaaggagaaggagaaggagaaggagaaggaaaaagagaaggaaaaggaaaaggagaaagaaaaggagaaagagaaagagaaggaaaaggagaaagagaaggagaaggaaaaagaaaaggagaaggaaaaggagaaggaaaaggagaaggagaaggaaaaggaaaaagagaaggagaaggagaaggagaaggagaaggagaaggagaaggagaaggagaaggagaaggagaaggagaaggagaaggagaaggagaaggagaaggaaaaagagaaggaaaaggaaaaggagaaagaaaaggagaaagagaaagagaaggaaaaggagaaagagaaggagaaggaaaaagaaaaggagaaggaaaaggagaaggaaaaggagaaggagaaggaaaaggaaaaagagaaggagaaggagaaggagaaggagaaggagaaggagaaggagaaggagaaggagaaggagaaggagaaggagaaggagaaggagaagtgcgacctgccacagacccactctctgggcagccggagaacactgatgctgctggcccagatgaggaagatctccctgttctcttgtctgaaggaccgccacgatttcggctttccccaggaggagttcggcaaccagtttcagaaggccgagacaatccctgtgctgcacgagatgatccagcagatcttcaatctgttttccacaaaggatagctccgccgcatgggacgagacactgctggataagttttacacagagctgtatcagcagctgaacgacctggaggcatgcgtgatccagggagtgggagtgaccgagacaccactgatgaaggaggattctatcctggccgtgaggaagtacttccagcgcatcaccctgtacctgaaggagaagaagtatagcccatgtgcatgggaggtggtgcgggcagagatcatgagatcttttagcctgtccacaaatctgcaggagagcctgcggtccaaggag

SEQ ID NO: 12 - protein, 433 aa, Artificial sequence
  Synthetic
MDAMKRGLCCVLLLCGAVFVSPSASYPYDVPDYAEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKEKCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 13 - DNA, 1410 bp, Artificial sequence
  Synthetic
atggacgccatgaagaggggcctgtgctgcgtgctgctgctgtgcggagccgtgttcgtgagcccctccgcctcttacccatacgatgttccagattacgctgagaaaccaaaagagcctgaaaagccagagaagccaaaggagccaaaagagcccgagaagcctaaggagcctgagaagcctgagaagcccaaggagcctaaggagccagagaagcccaaggagcctgagaaacctgaaaaacctaaagaaccaaaagaacctgaaaaacctaaggaaccagagaaacctgaaaaaccaaaagaaccaaaagaacccgaaaaacctaaagaacctgagaaacctgaaaagcctaaggaaccaaaagaacctgagaaaccaaaggagcctgagaagcccgaaaagcctaaggaacccaaagaacctgaaaaaccaaaggaacctgagaaacctgagaagccaaaagaaccaaaagagcctgagaagccaaaagagccagaaaaacctgaaaaacccaaagaaccaaaagaaccagaaaaacctaaagagccagaaaaacccgaaaaacctaaggaacccaaagagcctgaaaaacctaaagagcccgaaaaacctgaaaagccaaaagaaccaaaggaacccgaaaagccaaaagaacctgagaagcccgagaaacctaaagaaccaaaggaaccagaaaaacctaaggaacctgagaaacccgaaaaaccaaaagaacccaaagaacccgaaaaaccaaaggagccagagaaacctgaaaagccaaaggaaccaaaagaacccgagaaacctaaagagccagagaaacctgagaagcctaaagaacctaaggagcccgaaaagccaaaggaacccgagaaaccagaaaagcctaaagagcctaaagaaccaaaatgcgacctgccacagacccactctctgggcagccggagaacactgatgctgctggcccagatgaggaagatctccctgttctcttgtctgaaggaccgccacgatttcggctttccccaggaggagttcggcaaccagtttcagaaggccgagacaatccctgtgctgcacgagatgatccagcagatcttcaatctgttttccacaaaggatagctccgccgcatgggacgagacactgctggataagttttacacagagctgtatcagcagctgaacgacctggaggcatgcgtgatccagggagtgggagtgaccgagacaccactgatgaaggaggattctatcctggccgtgaggaagtacttccagcgcatcaccctgtacctgaaggagaagaagtatagcccatgtgcatgggaggtggtgcgggcagagatcatgagatcttttagcctgtccacaaatctgcaggagagcctgcggtccaaggag

SEQ ID NO: 14 - protein, 470 aa, Artificial sequence
  Synthetic
MDAMKRGLCCVLLLCGAVFVSPSASYPYDVPDYAEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPEKPKEPEKPEKPKEPKEPKCDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 15 - DNA, 597 bp, Artificial sequence
  Synthetic
atggacgccatgaagaggggcctgtgctgcgtgctgctgctgtgcggagccgtgttcgtgagcccctccgcctcttacccatacgatgttccagattacgcttgcgacctgccacagacccactctctgggcagccggagaacactgatgctgctggcccagatgaggaagatctccctgttctcttgtctgaaggaccgccacgatttcggctttccccaggaggagttcggcaaccagtttcagaaggccgagacaatccctgtgctgcacgagatgatccagcagatcttcaatctgttttccacaaaggatagctccgccgcatgggacgagacactgctggataagttttacacagagctgtatcagcagctgaacgacctggaggcatgcgtgatccagggagtgggagtgaccgagacaccactgatgaaggaggattctatcctggccgtgaggaagtacttccagcgcatcaccctgtacctgaaggagaagaagtatagcccatgtgcatgggaggtggtgcgggcagagatcatgagatcttttagcctgtccacaaatctgcaggagagcctgcggtccaaggag

SEQ ID NO: 16 - protein, 199 aa, Artificial sequence
  Synthetic
MDAMKRGLCCVLLLCGAVFVSPSASYPYDVPDYACDLPQTHSLGSRRTLMLLAQMRKISLFSCLKDRHDFGFPQEEFGNQFQKAETIPVLHEMIQQIFNLFSTKDSSAAWDETLLDKFYTELYQQLNDLEACVIQGVGVTETPLMKEDSILAVRKYFQRITLYLKEKKYSPCAWEVVRAEIMRSFSLSTNLQESLRSKE

SEQ ID NO: 17 - DNA, 990 bp, Artificial sequence
  Synthetic
atggttagcaaaggcgaggaactgttcaccggtgtggttccgatcctggtggagctggacggcgatgttaacggtcacaagtttagcgtgagcggcgagggcgaaggtgacgcgacctacggcaagctgaccctgaaattcatttgcaccaccggtaaactgccggtgccgtggccgaccctggttaccaccctgacctacggtgttcagtgctttagccgttatccggaccacatgaagcaacacgatttctttaaaagcgcgatgccggagggctacgtgcaggaacgtaccatcttctttaaggacgatggtaactataaaacccgtgcggaagtgaagttcgaaggcgacaccctggttaaccgtatcgagctgaagggtattgactttaaagaagatggcaacattctgggtcacaagctggagtacaactataacagccacaacgtgtacatcatggcggataagcagaaaaacggcatcaaggttaacttcaagatccgtcacaacattgaagacggtagcgtgcaactggcggatcactaccagcaaaacaccccgattggtgatggtccggttctgctgccggataaccactatctgagcacccaaagcgcgctgagcaaggacccgaacgagaaacgtgatcacatggtgctgctggaattcgttaccgcggcgggcattaccctgggtatggatgaactgtataaaaagcttgcggccgcactcgagaagcttgagaaggagaaggagaaggaaaaggagaaggagaaagagaaggagaaggagaaagaaaaggagaaggaaaaggaaaaggagaaggaaaaagagaaggagaaggaaaaagaaaaggagaaagagaaggaaaaggagaaagagaaagagaaggagaaagagaaagaaaaggagaaagaaaaggacaaggagaaagaaaaagagaaggagaaagaaaaagaaaaggagaaagaaaaagaaaaagaaaaa

SEQ ID NO: 18 - protein, 330 aa, Artificial sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKLAAALEKLEKEKEKEKEKEKEKEKEKEKEKEKEKEKEKEKEKEKEKEKEKEKEKEKEKEKEKEKEKEKDKEKEKEKEKEKEKEKEKEKEK

SEQ ID NO: 19 - DNA, 1038 bp, Artificial sequence
  Synthetic
atggttagcaaaggcgaggaactgttcaccggtgtggttccgatcctggtggagctggacggcgatgttaacggtcacaagtttagcgtgagcggcgagggcgaaggtgacgcgacctacggcaagctgaccctgaaattcatttgcaccaccggtaaactgccggtgccgtggccgaccctggttaccaccctgacctacggtgttcagtgctttagccgttatccggaccacatgaagcaacacgatttctttaaaagcgcgatgccggagggctacgtgcaggaacgtaccatcttctttaaggacgatggtaactataaaacccgtgcggaagtgaagttcgaaggcgacaccctggttaaccgtatcgagctgaagggtattgactttaaagaagatggcaacattctgggtcacaagctggagtacaactataacagccacaacgtgtacatcatggcggataagcagaaaaacggcatcaaggttaacttcaagatccgtcacaacattgaagacggtagcgtgcaactggcggatcactaccagcaaaacaccccgattggtgatggtccggttctgctgccggataaccactatctgagcacccaaagcgcgctgagcaaggacccgaacgagaaacgtgatcacatggtgctgctggaattcgttaccgcggcgggcattaccctgggtatggatgaactgtataaaaagcttgcggccgcactcgaggaaaagggtagcaacgaaaagggctctaatgagaagggctctaacgaaaaaggttctaacgaaaagggctccaatgaaaagggcagcaacgaaaaaggtagcaacgagaaaggcagcaatgagaaaggctctaacgagaagggcagcaatgaaaaaggcagcaacgagaagggttccaatgaaaaaggctccaacgagaagggttctaacgagaaaggttccaatgagaagggtagcaatgaaaagggttctaatgagaaaggtagcaatgaaaaaggttccaacgaaaaaggctctaac

SEQ ID NO: 20 - protein, 346 aa, Artificial sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKLAAALEEKGSNEKGSNEKGSNEKGSNEKGSNEKGSNEKGSNEKGSNEKGSNEKGSNEKGSNEKGSNEKGSNEKGSNEKGSNEKGSNEKGSNEKGSNEKGSNEKGSN

SEQ ID NO: 21 - DNA, 1026 bp, Artificial sequence
  Synthetic
atggttagcaaaggcgaggaactgttcaccggtgtggttccgatcctggtggagctggacggcgatgttaacggtcacaagtttagcgtgagcggcgagggcgaaggtgacgcgacctacggcaagctgaccctgaaattcatttgcaccaccggtaaactgccggtgccgtggccgaccctggttaccaccctgacctacggtgttcagtgctttagccgttatccggaccacatgaagcaacacgatttctttaaaagcgcgatgccggagggctacgtgcaggaacgtaccatcttctttaaggacgatggtaactataaaacccgtgcggaagtgaagttcgaaggcgacaccctggttaaccgtatcgagctgaagggtattgactttaaagaagatggcaacattctgggtcacaagctggagtacaactataacagccacaacgtgtacatcatggcggataagcagaaaaacggcatcaaggttaacttcaagatccgtcacaacattgaagacggtagcgtgcaactggcggatcactaccagcaaaacaccccgattggtgatggtccggttctgctgccggataaccactatctgagcacccaaagcgcgctgagcaaggacccgaacgagaaacgtgatcacatggtgctgctggaattcgttaccgcggcgggcattaccctgggtatggatgaactgtataaaaagcttgcggccgcactcgaggagaagggtgaaaaaggcgaaaaaggtgaaaaaggcgaaaagggcgaaaaaggcgaaaagggtgagaaaggcgaaaagggtgaaaagggcgaaaagggtgaaaaaggtgaaaagggtgagaagggcgagaaaggcgaaaaaggcgagaaaggtgagaaaggcgagaaaggtgaaaagggcgagaaaggtgaaaaaggtgagaaaggtgagaagggcgagaagggcgagaagggtgaaaagggtgagaaaggtgaaaaaggcgagaagggtgagaagggtgagaagggc

SEQ ID NO: 22 - protein, 342 aa, Artificial sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKLAAALEEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKGEKG

SEQ ID NO: 23 - DNA, 1038 bp, Artificial sequence
  Synthetic
atggttagcaaaggcgaggaactgttcaccggtgtggttccgatcctggtggagctggacggcgatgttaacggtcacaagtttagcgtgagcggcgagggcgaaggtgacgcgacctacggcaagctgaccctgaaattcatttgcaccaccggtaaactgccggtgccgtggccgaccctggttaccaccctgacctacggtgttcagtgctttagccgttatccggaccacatgaagcaacacgatttctttaaaagcgcgatgccggagggctacgtgcaggaacgtaccatcttctttaaggacgatggtaactataaaacccgtgcggaagtgaagttcgaaggcgacaccctggttaaccgtatcgagctgaagggtattgactttaaagaagatggcaacattctgggtcacaagctggagtacaactataacagccacaacgtgtacatcatggcggataagcagaaaaacggcatcaaggttaacttcaagatccgtcacaacattgaagacggtagcgtgcaactggcggatcactaccagcaaaacaccccgattggtgatggtccggttctgctgccggataaccactatctgagcacccaaagcgcgctgagcaaggacccgaacgagaaacgtgatcacatggtgctgctggaattcgttaccgcggcgggcattaccctgggtatggatgaactgtataaaaagcttgcggccgcactcgaggagaagggtagcgaaaaaggcagcgaaaaaggtagcgagaagggcagcgaaaagggcagcgagaagggcagcgagaaaggcagcgagaagggtagcgagaaaggcagcgaaaagggtagcgagaaaggatctgagaagggctctgaaaaaggtagcgagaaaggtagcgaaaaaggtagcgaaaagggctctgaaaaaggatctgaaaagggtagcgagaagggtagcgaaaagggatccgaaaaaggaagtgagaagggttctgaaaagggctctgaaaagggttctgagaagggtagc

SEQ ID NO: 24 - protein, 346 aa, Artificial sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKLAAALEEKGSEKGSEKGSEKGSEKGSEKGSEKGSEKGSEKGSEKGSEKGSEKGSEKGSEKGSEKGSEKGSEKGSEKGSEKGSEKGSEKGSEKGSEKGSEKGSEKGS

SEQ ID NO: 25 - DNA, 738 bp, Artificial sequence
  Synthetic
atggttagcaaaggcgaggaactgttcaccggtgtggttccgatcctggtggagctggacggcgatgttaacggtcacaagtttagcgtgagcggcgagggcgaaggtgacgcgacctacggcaagctgaccctgaaattcatttgcaccaccggtaaactgccggtgccgtggccgaccctggttaccaccctgacctacggtgttcagtgctttagccgttatccggaccacatgaagcaacacgatttctttaaaagcgcgatgccggagggctacgtgcaggaacgtaccatcttctttaaggacgatggtaactataaaacccgtgcggaagtgaagttcgaaggcgacaccctggttaaccgtatcgagctgaagggtattgactttaaagaagatggcaacattctgggtcacaagctggagtacaactataacagccacaacgtgtacatcatggcggataagcagaaaaacggcatcaaggttaacttcaagatccgtcacaacattgaagacggtagcgtgcaactggcggatcactaccagcaaaacaccccgattggtgatggtccggttctgctgccggataaccactatctgagcacccaaagcgcgctgagcaaggacccgaacgagaaacgtgatcacatggtgctgctggaattcgttaccgcggcgggcattaccctgggtatggatgaactgtataaaaagcttgcggccgcactcgag

SEQ ID NO: 26 - protein, 246 aa, Artificial sequence
  Synthetic
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYKKLAAALE